(19) United States
(12) Patent Application Publication
(10) Pub. No.: US 2006/0019258 A1
(43) Pub. Date: Jan. 26, 2006

(54) Methods and compositions for detection of small interfering RNA and micro-RNA

(75) Inventor: Joanne M. Yeakley, Encinitas, CA (US)

Correspondence Address: ILLUMINA, INC., 9885 TOWNE CENTRE DRIVE, SAN DIEGO, CA, 92121-1975, US

(73) Assignee: Illumina, Inc., San Diego, CA (US)

(21) Appl. No.: 10/894,969

(22) Filed: Jul. 20, 2004

(51) Int. Cl. C12Q 1/68; C07H 21/02

(52) U.S. Cl. 435/6; 536/25.32

(57) ABSTRACT

The invention provides a method of distinguishing small RNA from mRNA by contacting a biological isolate with a phosphate reactive reagent having a label moiety under conditions wherein the label moiety is preferentially added to the 5′ phosphate of small RNA over the 5′ cap structure of mRNA and distinguishing the small RNA from the mRNA according to the presence of the label. The invention further provides a method of identifying a plurality of different small RNAs by adding a unique extension sequences to different small RNA sequences and identifying the extended small RNA sequences. Furthermore, the invention provides diagnostic methods for determining presence of a disease or condition such as cancer. Also provided are prognostic methods for determining progression of a disease or condition or for monitoring effectiveness of a treatment for a disease or condition

This invention was made with government support under grant number R21 CA88351 awarded by the National Institute of Health. The United States Government has certain rights in this invention.

### BACKGROUND OF THE INVENTION

This invention relates generally to detection of nucleic acids, and more specifically to detection of small RNA such as small interfering RNA (siRNA) and micro-RNA (miRNA).

Small interfering RNA and miRNA have recently become the subjects of intense research interest in biology and medicine due to their apparent roles in the regulation of gene expression via a process termed RNA interference (RNAi). The ability of organisms to dynamically respond to their environment is due in large part to regulation of gene expression. Regulation of gene expression is also important for the ability of multicellular organisms to generate the proper type and number of cells to create complex tissues and organs at the appropriate locations and times during development. Control of gene expression by a cell requires perception of environmental signals and appropriate response to these signals. Proteins have been studied extensively as mediators of these signals and a large number of protein-based regulators of gene expression are known. In contrast, the process of RNAi and, in particular, the role of siRNA and miRNA in regulating gene expression is just beginning to be elucidated.

Micro-RNA molecules are produced as cleavage products of larger precursors that form self-complementary hairpin structures. The miRNA molecules are typically 21 or 22 nucleotides in length and are processed by a ribonuclease (such as Dicer in animals and DICER-LIKE1 in plants). A miRNA precursor can by polycistronic containing several different hairpin structures that each give rise to a different miRNA molecule. Small interfering RNA molecules are also generally about 21 or 22 nucleotides long but, on the other hand, are produced from long hairpin precursors processed such that several different siRNA molecules can arise from a single hairpin structure.

Typically, miRNA hybridizes to a specific target mRNA through near complementary base pairing to form large complexes. Complex formation results in arrest of translation and/or increased degradation of the target mRNA. siRNAs have been found to associate with an RNA-induced silencing complex (RISC) to guide sequence-specific cleavage of mRNA. Interestingly, miRNAs and siRNAs have been found to be functionally interchangeable, operating in either of these pathways.

To date, most siRNAs and miRNAs have been identified by cloning techniques. A few miRNAs have been identified by positional cloning methods—a method which can be quite time consuming. The majority have been cloned from size fractionated RNA samples. A problem with using size fractionated samples is that other RNA contaminants such as mRNA degradation products, short ribosomal RNAs, and tRNAs are also cloned from size-fractionated samples, which can render identification of true miRNAs and siRNAs difficult.

Thus, there exists a need for methods of isolating siRNA and miRNA. There also exists a need for methods of detecting the diversity of siRNA and miRNA in a cell or organism. The present invention satisfies this need and provides other advantages as well.

### BRIEF SUMMARY OF THE INVENTION

The invention provides a method of distinguishing small RNA from mRNA. The method includes the steps of (a) providing a biological isolate including mRNA having a 5′ cap structure and small RNA having a 5′ phosphate; (b) contacting the isolate with a phosphate reactive reagent having a label moiety under conditions wherein the label moiety is preferentially added to the 5′ phosphate over the 5′ cap structure, thereby producing labeled small RNA; and (c) distinguishing the small RNA from the mRNA according to the presence of the label.

The invention further provides a method of identifying a plurality of different small RNAs. The method includes the steps of (a) providing a plurality of different small RNA sequences; (b) adding unique extension sequences to the different small RNA sequences, thereby forming a plurality of extended small RNA sequences; and (c) detecting the extended small RNA sequences, thereby identifying the plurality of different small RNAs.

Also provided is a method of detecting a plurality of different small RNAs. The method includes the steps of (a) providing a biological isolate including mRNA having a 5′ cap structure and a plurality of different small RNA molecules having a 5′ phosphate; (b) contacting the mixture with a phosphate reactive reagent having a label moiety under conditions wherein the label moiety is preferentially added to the 5′ phosphate over the 5′ cap structure, thereby producing a plurality of labeled small RNA; (c) adding a unique extension sequence to each different small RNA, thereby forming a plurality of extended small RNAs; and (d) detecting the extended small RNAs, thereby identifying the plurality of different small RNAs.

The invention provides methods for diagnosing the occurrence of cancer in a patient at risk for cancer. The method involves (a) measuring a level of one or more small RNAs in a neoplastic cell-containing sample from patient at risk for cancer, and (b) comparing the level of the one or more small RNAs in the sample to a reference level, wherein a different level of the one or more small RNAs in the sample correlates with presence of cancer in the patient.

The invention also provides methods for determining a prognosis for survival for a cancer patient. One method involves (a) measuring a level of one or more small RNAs in a neoplastic cell-containing sample from the cancer patient, and (b) comparing the level of the one or more small RNAs in the sample to a reference level, wherein a different level of the one or more small RNAs in the sample correlates with increased survival of the patient.

The invention also provides a method for monitoring the effectiveness of a course of treatment for a patient with cancer. The method involves (a) determining a level of one or more small RNAs in a neoplastic cell containing sample from the cancer patient prior to treatment, and (b) determining the level of one or more small RNAs in a neoplastic cell-containing sample from the patient after treatment, whereby comparison of the level of one or more small RNAs prior to treatment with the level of one or more small RNAs after treatment indicates the effectiveness of the treatment.

### BRIEF DESCRIPTION OF THE DRAWINGS

FIG. 1 shows a diagrammatic representation of one embodiment of the invention for adding an extension sequence to a small RNA molecule

### DETAILED DESCRIPTION OF THE INVENTION

This invention provides a method of distinguishing small RNA molecules, typically involved in RNA interference (RNAi), from other cellular nucleic acids such as other RNAs. The invention exploits structural features of short RNA that are unique compared to other cellular nucleic acids such as mRNA. In particular, short RNA has an underivatized 5′ phosphate which is unique compared to messenger RNA (mRNA) which has a cap structure at the 5′ end. An advantage of the invention is that the ability to distinguish short RNAs from mRNA improves analysis and evaluation of RNAi in research and clinical settings by reducing artifacts that can arise from the presence of unwanted contaminants.

The invention further provides a method of identifying a plurality of different short RNA molecules in a biological isolate. Many nucleic acid assays are compromised or precluded from use when targets are the size of small RNAs. Furthermore, many small RNAs have similar sequences making it difficult to differentiate different molecules from each other using standard hybridization based assays. Methods are provided herein for adding sequence specificity and length to small RNA sequences and detecting the extended small RNA sequences. An advantage of the methods is that several small RNA sequences can be simultaneously detected, thereby allowing the use of multiplex methods in which the diversity of small RNA sequences present in a cell or organism can be readily evaluated in a research, or clinical setting.

Definitions

As used herein, the term “small RNA” is intended to mean a ribonucleic acid having a length between about 20 and 30 nucleotides, and terminating in a 5′ phosphate and a 3′ hydroxyl. A 5′ phosphate is understood to be a (PO₄)²⁻ (PO₄H)⁻ or (PO₄H₂) moiety covalently attached to the 5′ carbon of ribose via one of the oxygens. A 3′ hydroxyl is understood to be an OH or O⁻ moiety covalently attached to the 3′ carbon of ribose via the oxygen. Those skilled in the art will recognize that the presence or absence of hydrogens in the phosphate and hydroxyl moieties as listed above is a function of their pKa values and the pH of their environment. Most small RNA molecules are 20 to 25 nucleotides in length with a large majority being about 21 or 22 nucleotides long. However, small RNA molecules having longer sequences are also known including for example, those having a length of 26 nucleotides (see, for example, Hamilton et al., EMBO J. 21:4671 (2002)) or 28 nucleotides (see, for example, Mochizuki et al., Cell 110:689-99 (2002)).

Small RNA can be identified according to its function in a cell including, for example, having a non-coding sequence (i.e. not being translated into protein) and being capable of inhibiting expression of at least one mRNA. Small RNA can also be identified according to its biosynthesis. For example, a first type of small RNA, short interfering RNA (siRNA), is typically synthesized from endogenous or exogenous double stranded RNA (dsRNA) molecules having hairpin structures and processed such that numerous siRNA molecules are produced from both strands of the hairpin. In contrast, micro-RNA molecules are typically produced from endogenous dsRNA molecules having one or more hairpin structure such that a single micro-RNA molecule is produced from each hairpin structure. The terms “small RNA,” “siRNA” and “micro-RNA” are intended to be consistent with their use in the art as described, for example, in Ambros et al., RNA 9:277-279 (2003).

A small RNA can be distinguished from mRNA based on the presence of a 5′ cap structure in mRNA and absence of the cap structure in small RNA. The 5′ cap structure typically found in eukaryotic mRNA is a 7-methylguanylate having a 5′ to 5′ triphosphate linkage to the terminal nucleotide. Small RNA can also be distinguished from mRNA based on the presence of a terminal polyadenylate sequence at the 3′ end of mRNA which is absent in small RNA.

As used herein, the term “biological isolate” is intended to mean one or more substances removed from at least one co-occurring molecule of an organism. An isolated nucleic acid can, for example, be essentially free of other nucleic acids such that it is increased to a significantly higher fraction of the total nucleic acid present in the biological isolate than in the cells from which it was taken. For example, an isolated nucleic acid can be enriched at least 2, 5, 10, 50, 100, 1000 fold or higher in the biological isolate compared to in the cell from which it was taken. A biological isolate can be obtained from an intact organism, tissue or cell. Exemplary eukaryotes from which biological isolates can be derived in a method of the invention include, without limitation, a mammal such as a rodent, mouse, rat, rabbit, guinea pig, ungulate, horse, sheep, pig, goat, cow, cat, dog, primate, human or non-human primate; a plant such as Arabidopsis thaliana, corn (Zea mays), sorghum, oat (oryza sativa), wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish (Danio rerio); a reptile; an amphibian such as a frog or Xenopus laevis; a dictyostelium discoideum; a fungi such as pneumocystis carinii, Takifugu rubripes, yeast, Saccharamoyces cerevisiae or Schizosaccharomyces pombe; or a plasmodium falciparum. In addition to animal and plant systems, the invention can be used with a prokaryote system including, for example, a bacterium such as Escherichia coli, staphylococci or mycoplasma pneumoniae; an archae; a virus such as Hepatitis C virus or human immunodeficiency virus; or a viroid. Endogenous small RNA can be isolated from a biological system from which it was synthesized. Exogenous small RNA can be isolated from a biological system from which it was transmitted, for example, by viral infection or treatment with a small RNA precursor. Exemplary small RNA precursors include double stranded RNAs such as those described in further detail herein below.

As used herein, the term “phosphate reactive” is intended to mean capable of covalently modifying a phosphate by addition of a moiety. An exemplary phosphate reactive reagent is an activator and a label mòiety having a group that is reactive with phosphate in the presence of the activator. Exemplary activators include, but are not limited to, various carbodiimides, cyanogens bromide; imidazole and its derivatives; N-hydroxybenzotriazole; coupling reagents normally used in peptide synthesis such as benzotriazole-1-yl-oxy-tris-(dimethylamino)-phosphoniumihexafluorophosphate; 1,1′-Carbonyl-diimidazole; Di-(N-Succinimidyl)carbonate; 2-(1H-Benzotriazole-1-yl)-1,1,3,3-tetramethyluronium hexafluorophosphate; 1-(Mesitylene-2-sulfonyl)-3-nitro-1H-1,2,4-triazole; Benzotriazole-1-yl-oxy-tris-pyrrolidino-phosphonium hexafluorophosphate; 2-(1H-Benzotriazole-1-yl)-1,1,3,3-tetramethyluronium tetrafluoroborate. A label moiety can have a reactive group such as an amine, hydroxyl, hydrazine, hydrazide, thiosemicarbazide, thiol or phosphate.

As used herein, the term “label moiety” is intended to mean one or more atom that can be specifically detected to identify a substance to which the one or more atom is attached. A label moiety can be a primary label that is directly detectable or secondary label that can be indirectly detected, for example, via interaction with a primary label. Exemplary primary labels include, without limitation, an isotopic label such as a naturally non-abundant heavy isotope or radioactive isotope examples of which include ¹⁴C, ¹²³I, ¹²⁴I, ¹²⁵I, ¹³¹I, ³²P, ³⁵S or ³H; optically detectable moieties such as a chromophore, luminophore, fluorophore, quantum dot or nanoparticle light scattering label; electromagnetic spin label; calorimetric agent; magnetic substance; electron-rich material such as a metal; electrochemiluminescent label such as Ru(bpy)₃²⁺; moiety that can be detected based on a nuclear magnetic, paramagnetic, electrical, charge to mass, or thermal characteristic; or light scattering or plasmon resonant materials such as gold or silver particles. Fluorophores that are useful in the invention include, for example, fluorescent lanthanide complexes, including those of Europium and Terbium, fluorescein, fluorescein isothiocyanate, dichlorotriazinylamine fluorescein, rhodamine, tetramethylrhodamine, umbelliferone, eosin, erythrosin, coumarin, methyl-coumarins, pyrene, Malacite green, Cy3, Cy5, stilbene, Lucifer Yellow, Cascade Blue™, Texas Red, alexa dyes, dansyl chloride, phycoerythin, green fluorescent protein and its wavelength shifted variants, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066.

Exemplary secondary labels are binding moieties such as a receptor, ligand or other member of a pair of molecules having binding specificity for each other. Exemplary binding moieties having specificity for each other include, without limitation, streptavidin/biotin, avidin/biotin or an antigen/antibody complex such as rabbit IgG and anti-rabbit IgG. Specific affinity between two binding partners is understood to mean preferential binding of one partner to another compared to binding of the partner to other components or contaminants in the system. Binding partners that are specifically bound typically remain bound under the detection or separation conditions described herein, including wash steps to remove non-specific binding. Depending upon the particular binding conditions used, the dissociation constants of the pair can be, for example, less than about 10⁻⁴, 10⁻⁵, 10⁻⁶, 10⁻⁷, 10⁻⁸, 10⁻⁹, 10⁻¹⁰, 10⁻¹¹, or 10⁻¹² M⁻¹. Secondary labels also include enzymes that produce a detectable product such as horseradish peroxidase, alkaline phosphatase, β-galactosidase, or acetylcholinesterase.

The terms “receptor” and “ligand” are used herein for clarity in identifying binding partners. Accordingly, the term “receptor” is intended to mean a molecule that is capable of selectively binding a ligand and the term “ligand” is intended to mean a molecule that is capable of selectively binding a receptor. The terms are intended to encompass receptors or ligands that have other functions as well. However, the terms are not intended to be limited by any other function unless indicated otherwise. For example, a receptor can be a naturally occurring polypeptide having signal transducing activity or a functional fragment or modified form of the entire polypeptide that exhibits selective binding to a ligand whether or not the functional fragment has signal transducing activity.

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, an array can include separate substrates each bearing a different probe molecule. Probes attached to separate substrates can be identified according to the locations of the substrates on a surface to which the substrates are associated or according to the locations of the substrates in a liquid. Exemplary arrays in which separate substrates are located on a surface include, without limitation, those including beads in wells. Arrays useful in the invention are described, for example, in U.S. Pat. Nos. 6,023,540, 6,200,737, 6,327,410, 6,355,431 and 6,429,027; U.S. patent application publication No. U.S. 2002/0102578 and PCT Publication Nos. WO 00/63437, WO 98/40726, and WO 98/50782. Further examples of arrays that can be used in the invention are described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897. Commercially available fluid formats for distinguishing beads include, for example, those used in xMAP™ technologies from Luminex or MPSS™ methods from Lynx Therapeutics.

### Description of Particular Embodiments

The invention provides a method of distinguishing small RNA from mRNA. The method includes the steps of (a) providing a biological isolate including mRNA having a 5′ cap structure and small RNA having a 5′ phosphate; (b) contacting the isolate with a phosphate reactive reagent having a label moiety under conditions wherein the label moiety is preferentially added to the 5′ phosphate over the 5′ cap structure, thereby producing labeled small RNA; and (c) distinguishing the small RNA from the mRNA according to the presence of the label.

A biological isolate used in the invention can be from any of a variety of organisms including, without limitation, those set forth above. In many cases, useful biological isolates are available from commercial sources or from banks and depositories administered by public or private institutions such as the American Type Culture Collection (ATCC). For many applications, it is desirable that isolation protocols used by commercial sources are not biased against retention of small RNAs of interest for a particular application of the invention. A biological isolate can be from one or more cells, bodily fluids or tissues. Known methods can be used to obtain a bodily fluid such as blood, sweat, tears, lymph, urine, saliva, semen, cerebrospinal fluid, feces or amniotic fluid. Similarly known biopsy methods can be used to obtain cells or tissues such as buccal swab, mouthwash, surgical removal, biopsy aspiration or the like. A biological isolate can also be obtained from one or more cell or tissue in primary culture, in a propagated cell line, a fixed archival sample, forensic sample or archeological sample.

Exemplary cell types from which a nucleic acid-containing isolate can be obtained in a method of the invention include, without limitation, a blood cell such as a B lymphocyte, T lymphocyte, leukocyte, erythrocyte, macrophage, or neutrophil; a muscle cell such as a skeletal cell, smooth muscle cell or cardiac muscle cell; germ cell such as a sperm or egg; epithelial cell; connective tissue cell such as an adipocyte, fibroblast or osteoblast; neuron; astrocyte; stromal cell; kidney cell; pancreatic cell; liver cell; or keratinocyte. A cell from which an isolate is obtained can be at a particular developmental level including, for example, a hematopoietic stem cell or a cell that arises from a hematopoietic stem cell such as a red blood cell, B lymphocyte, T lymphocyte, natural killer cell, neutrophil, basophil, eosinophil, monocyte, macrophage, or platelet. Other cells include a bone marrow stromal cell (mesenchymal stem cell) or a cell that develops therefrom such as a bone cell (osteocyte), cartilage cells (chondrocyte), fat cell (adipocyte), or other kinds of connective tissue cells such as one found in tendons; neural stem cell or a cell it gives rise to including, for example, a nerve cell (neuron), astrocyte or oligodendrocyte; epithelial stem cell or a cell that arises from an epithelial stem cell such as an absorptive cell, goblet cell, Paneth cell, or enteroendocrine cell; skin stem cell; epidermal stem cell; or follicular stem cell. Generally, any type of stem cell can be used including, without limitation, an embryonic stem cell, adult stem cell, or pluripotent stem cell.

A cell from which an isolate is obtained for use in the invention can be a normal cell or a cell displaying one or more symptom of a particular disease or condition. Thus, a biological isolate used in a method of the invention can be obtained from a cancer cell, neoplastic cell, necrotic cell or cell experiencing a disease or condition set forth below. Those skilled in the art will know or be able to readily determine methods for isolating samples from a cell, fluid or tissue using methods known in the art such as those described in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998).

A method of the invention can further include steps of isolating a particular type of cell or tissue. Exemplary methods that can be used in a method of the invention to isolate a particular cell from other cells in a population include, but are not limited to, Fluorescent Activated Cell Sorting (FACS) as described, for example, in Shapiro, Practical Flow Cytometry, 3rd edition Wiley-Liss; (1995), density gradient centrifugation, or manual separation using micromanipulation methods with microscope assistance. Exemplary cell separation devices that are useful in the invention include, without limitation, a Beckman JE-6 centrifugal elutriation system, Beckman Coulter EPICS ALTRA computer-controlled Flow Cytometer-cell sorter, Modular Flow Cytometer from Cytomation, Inc., Coulter counter and channelyzer system, density gradient apparatus, cytocentrifuge, Beckman J-6 centrifuge, EPICS V dual laser cell sorter, or EPICS PROFILE flow cytometer. A tissue or population of cells can also be removed by surgical techniques. For example, a tumor or cells from a tumor can be removed from a tissue by surgical methods, or conversely non-cancerous cells can be removed from the vicinity of a tumor. Using methods such as those set forth in further detail below, the invention can be used to compare the type or amount of small RNA present in different cells including, for example, cancerous and non-cancerous cells isolated from the same individual or from different individuals.

A biological isolate can be prepared for use in a method of the invention by lysing a cell that contains one or more desired nucleic acids. Typically, a cell is lysed under conditions that substantially preserve the integrity of the desired nucleic acid. For example, cells can be lysed or subfractions obtained under conditions that stabilize RNA integrity. Such conditions include, for example, cell lysis in strong denaturants, including chaotropic salts such as guanidine thiocyanate, ionic detergents such as sodium dodecyl sulfate, organic solvents such as phenol, high lithium chloride concentrations or other conditions known in the art to be effective in limiting the activity of endogenous RNases during RNA purification as described, for example, in Sambrook et al., supra (2001) or in Ausubel et al., supra (1998). Additionally, relatively undamaged nucleic acids such as RNA can be obtained from a cell lysed by an enzyme that degrades the cell wall. Cells lacking a cell wall either naturally or due to enzymatic removal can also be lysed by exposure to osmotic stress. Other conditions that can be used to lyse a cell include exposure to detergents, mechanical disruption, sonication, heat, pressure differential such as in a French press device, or Dounce homogenization.

Agents that stabilize nucleic acids can be included in a cell lysate or other biological isolate including, for example, nuclease inhibitors such as ribonucleases inhibitors or deoxyribonuclease inhibitors, chelating agents, salts buffers and the like. Methods for lysing a cell to obtain nucleic acids can be carried out under conditions known in the art as described, for example, in Sambrook et al., supra (2001) or in Ausubel et al., supra, (1998).

In particular embodiments, a biological isolate used in a method of the invention can be a crude cell lysate obtained without further isolation of nucleic acids. Alternatively, a nucleic acid of interest can be further isolated from other cellular components in a method of the invention. In particular embodiments, a method of the invention can be carried out on purified or partially purified RNA. RNA can be isolated using known separation methods including, for example, liquid phase extraction, precipitation or solid phase extraction. Such methods are described, for example, in Sambrook et al., supra, (2001) or in Ausubel et al., supra, (1998) or available from various commercial vendors including, for example, Qiagen (Valencia, Calif.) or Promega (Madison, Wis.).

If desired, nucleic acids can be separated based on properties such as mass, charge to mass, or the presence of a particular sequence. Useful methods for separating nucleic acids include, but are not limited to, electrophoresis using agarose or polyacrylamide gels, capillary electrophoresis, conventional chromatography methods such as size exclusion chromatography, reverse phase chromatography or ion exchange chromatography or affinity methods such as affinity chromatography or precipitation using solid-phase poly dT oligonucleotides. Those skilled in the art will know or be able to determine an appropriate separation method or combination of separation methods to obtain a biological isolate of a desired nucleic acid composition and purity. In particular embodiments, proteins and large genomic DNA can be removed from RNA, for example, using precipitation and centrifugation methods that exploit the larger size of the genomic DNA and proteins. Messenger RNA can be removed from other RNA species, for example, using precipitation with poly dT oligonucleotide beads or size exclusion chromatography. Such methods can be used in combination with selective modification of the 5′ phosphate of small RNA to distinguish small RNA from other cellular components. Another useful method is differential elution of RNAs of different sizes from glass using washes of different ionic strength, an example of which is the mirVana™ miRNA Isolation Kit and protocol commercially available from Ambion (Austin, Tex.).

A method of the invention can include a step of contacting a biological isolate with a phosphate reactive reagent under conditions wherein the 5′ phosphate of small RNA is preferentially modified. A phosphate reactive reagent used in a method of the invention can include a label moiety or label precursor moiety such that 5′ phosphate modification produces a small RNA containing the label.

A useful reagent can preferentially add a moiety to a phosphate over one or more other molecules or moieties present in the same biological isolate or other reaction mixture. For example, a phosphate reactive agent can be added to a biological isolate that contains small RNA and mRNA under conditions in which it preferentially reacts with the 5′ phosphate of the small RNA but has reduced or insubstantial reactivity with mRNA having a 5′ cap structure. Thus, a phosphate reactive reagent that is useful in the invention can be inert to reaction with one or more other molecule or moiety in a biological isolate or reaction mixture including, for example, mRNA or a particular moiety of mRNA such as the 5′ cap structure. Useful reagents include those that are unreactive to the 3′ hydroxyl of nucleic acids.

A phosphate reactive reagent can be a single molecule or a combination of molecules. For example, a single molecule can contain a reactive moiety linked to a label moiety such that reaction between the reactive moiety and the 5′ phosphate of small RNA produces a small RNA linked to the label moiety.

In other embodiments, a combination of molecules can be used as a phosphate reactive reagent. For example, a first label molecule can be contacted with a biological isolate in the presence of a small RNA and a second molecule that activates the 5′ phosphate or the first label molecule, thereby producing a small RNA with an attached label. In a particular embodiment, the phosphate reagent can include a label moiety having a linked amino group and the second molecule can be a carbodiimide molecule that activates the 5′ phosphate to react with the amino group to produce a small RNA having a phosphoramidite linkage to the label moiety. Other exemplary phosphate reactive agents include, without limitation, ε-(6-(biotinoyl)amino)hexanoyl-L-lysine, hydrazide; DSB-X™ biotin hydrazide; DSB-X™ desthiobiocytin (-desthiobiotinoyl-L-lysine); DSB-X™ biotin ethylenediamine (desthiobiotin-X ethylenediamine, hydrochloride); Biotin-X cadaverine; Alexa Fluor® cadaverine; 5-(aminoacetamido)fluorescein(fluoresceinyl glycine amide); 4′-(aminomethyl)fluorescein, hydrochloride; 5-(((2-(carbohydrazino)methyl)thio) acetyl)aminofluorescein; fluorescein-5-thiosemicarbazide; N-methyl-4-hydrazino-7-nitrobenzofurazan; Oregon Green® 488 cadaverine; 5-((5-aminopentyl)thioureidyl)eosin, hydrochloride; Texas Red® cadaverine; Texas Red® hydrazide; bimane amine; poly(ethylene glycol) methyl ether, amine-terminated; and Lissamine™ rhodamine B ethylenediamine. Those skilled in the art will recognize that any of a variety of label moieties can be replaced for those listed in these reagents. For example, fluorescein can be replaced with other fluorophores described herein.

A short RNA can be distinguished from other components of a biological isolate, such as mRNA, rRNA, tRNA or other types of RNA, according to the presence of a label. Exemplary labels that can be used in the invention are set forth above and include primary and secondary labels. The label can be a primary label that is detected directly using methods such as those set forth below. A label can be a secondary label that is detected based on interaction with another reagent that produces a detectable label or is otherwise rendered detectable due to presence of the secondary label. For example, the secondary label can be a ligand and the ligand can be detected based on specific interaction with a receptor that is itself labeled or otherwise capable of being detected.

A small RNA that contains a label can be distinguished from other molecules that are devoid of the label using methods known in the art. Exemplary properties upon which detection can be based include, but are not limited to, mass, electrical conductivity or optical signals such as a fluorescent signal, absorption signal, luminescent signal, chemiluminescent signal or the like. Detection can also be based on absence or reduced level of one or more signal, for example, due to presence of a signal quenching moiety or degradation of a label moiety.

Detection of fluorescence can be carried out by irradiating a labeled nucleic acid with an excitatory wavelength of radiation and detecting radiation emitted from a fluorophore therein by methods known in the art and described, for example, in Lakowicz, Principles of Fluorescence Spectroscopy, 2nd Ed., Plenum Press New York (1999). A fluorophore can be detected based on any of a variety of fluorescence phenomena including, for example, emission wavelength, excitation wavelength, fluorescence resonance energy transfer (FRET) intensity, quenching, anisotropy or lifetime. FRET can be used to identify hybridization between a first polynucleotide attached to a donor fluorophore and a second polynucleotide attached to an acceptor fluorophore due to transfer of energy from the excited donor to the acceptor. Thus, hybridization can be detected as a shift in wavelength caused by reduction of donor emission and appearance of acceptor emission for the hybrid.

Other detection techniques that can be used to perceive or identify nucleic acids include, for example, mass spectrometry which can be used to perceive a nucleic acid based on mass; surface plasmon resonance which can be used to perceive a nucleic acid based on binding to a surface immobilized complementary sequence; absorbance spectroscopy which can be used to perceive a nucleic acid based on the wavelength of absorbed energy; calorimetry which can be used to perceive a nucleic acid based on changes in temperature of its environment upon binding to a complementary sequence; electrical conductance or impedance which can be used to perceive a nucleic acid based on changes in its electrical properties or in the electrical properties of its environment, magnetic resonance which can be used to perceive a nucleic acid based on presence of magnetic nuclei, or other known analytic spectroscopic or chromatographic techniques.

A labeled small RNA can be distinguished from one or more other cellular components by separating the labeled small RNA from the one or more other cellular components such as mRNA or other RNA species. If desired, the separated labeled small RNA can be further distinguished from other cellular components using detection methods such as those set forth above in regard to detecting primary labels. Secondary labels that can be attached to a small RNA and their binding partners that can be used for separation are set forth above. For example, a small RNA can be labeled with a ligand. A ligand labeled small RNA can be separated from other components of a biological isolate using a solid-phase immobilized receptor having binding specificity for the receptor. In other embodiments, the RNA-ligand:receptor complex can be precipitated using methods such as those employed for immunoprecipitation. Those skilled in the art will know or be able to determine appropriate affinity separation methods based on the particular binding partners used.

By way of example, a secondary label can be a hapten or antigen having affinity for an immunoglobulin, or functional fragment thereof. The immunoglobulin or functional fragment can be attached to a solid support. Labeled nucleic acids that are bound to the immunoglobulin can be separated from unlabeled nucleic acids by physical separation of the solid support and soluble fraction or in cases where the immunoglobulin is not bound to a solid support separation can be carried out by immunoprecipitation. In addition, avidin/biotin systems including, for example, those utilizing streptavidin, biotin or functional variants of each, can be used to separate modified nucleic acids from those that are unmodified. Typically the smaller of two binding partners is attached to a nucleic acid. However, attachment of the larger partner can also be useful. For example, the addition of streptavidin to a nucleic acid increases its size and changes its physical properties, which can be exploited for separation. Accordingly, a streptavidin labeled nucleic acid can be separated from unlabeled nucleic acids in a mixture using a technique such as size exclusion chromatography, affinity chromatography, filtration or differential precipitation.

In embodiments, including attachment of a binding partner to a solid support, the solid support can be selected, for example, from those described herein with respect to detection arrays. Particularly useful substrates include, for example, magnetic beads which can be easily introduced to the nucleic acid sample and easily removed with a magnet. Other known affinity chromatography substrates can be used as well. Known methods can be used to attach a binding partner to a solid support.

A label can be attached to a small RNA or other nucleic acid via a scissile linkage, if desired. Thus, a method of the invention can include a step of removing a label moiety from a small RNA. The label can be removed during or after distinguishing or detecting the small RNA as desired to suit a particular application of the methods. Removal of labels can be performed, for example, when unwanted during subsequent manipulations of an isolated small RNA. For example, removal of a label at the 5′ phosphate can be achieved prior to ligation of the 5′ phosphate to an extension sequence or probe in a method set froth below.

Exemplary scissile linkages that are useful include, but are not limited to, a photocleavable linkage such as ortho-nitrobenzyl groups, c-methylphenacyl ester; an enzymatically cleavable linkage such as a peptide recognized by a protease or a nucleotide sequence cleaved by a nuclease; acid or base labile linkages or linkages that are cleaved by specific chemicals. Enzymatically cleavable linkages can be recognized in a sequence specific fashion examples of which include polypeptides such as the prosequences of proteins and nucleic acids such as restriction endonuclease sites.

A method of the invention can be used to detect, identify or otherwise distinguish a plurality of small RNA molecules, having different sequences. In accordance with the methods set forth herein, a plurality of small RNA molecules can include at least 2, 5, 10, 50, 100, 500, 1,000, 5,000 or 10,000 different small RNA molecules up to and including the amount of different small RNA molecules found in a cell or population of cells being evaluated.

A plurality of small RNA molecules can be distinguished using an array of probe molecules. Exemplary arrays that can be used in the invention include, without limitation, those set forth previously herein. A probe can be any molecule or material that directly or indirectly binds a nucleic acid having a target sequence. A probe can be, for example, a nucleic acid that has a sequence that is complementary to a desired target nucleic acid or another molecule that binds to a nucleic acid in a sequence-specific fashion. Various techniques and technologies known in the art can be used for synthesizing arrays such as those set forth in further detail below. Furthermore, several array platforms are commercially available as set forth below.

In particular embodiments, probes useful for detecting small RNA molecules or other nucleic acids can be attached to particles that are arrayed or otherwise spatially distinguished. Particles useful in the invention are often referred to as microspheres or beads. However, such particles need not be spherical. Rather particles having other shapes including, but not limited to, disks, plates, chips, slivers or irregular shapes can be used. In addition, particles used in the invention can be porous, thus increasing the surface area available for attachment or assay of probe-fragment hybrids. Particle sizes can range, for example, from a few nanometers to many millimeters in diameter as desired for a particular application. For example, particles can be at least about 0.1 micron, 0.5 micron, 1 micron, 10 micron or 100 microns or larger in average diameter. The composition of the beads can vary depending, for example, on the application of the invention or the method of synthesis. Suitable bead compositions include, but are not limited to, those used in peptide, nucleic acid and organic moiety synthesis, such as plastics, ceramics, glass, polystyrene, methylstyrene, acrylic polymers, paramagnetic materials, thoria sol, carbon graphite, titanium dioxide, latex or cross-linked dextrans such as Sepharose™, cellulose, nylon, cross-linked micelles or Teflon™. Useful particles are described, for example, in Microsphere Detection Guide from Bangs Laboratories, Fishers Ind.

Several embodiments of array-based detection in the invention are exemplified below for beads or microspheres. Those skilled in the art will recognize that particles of other shapes and sizes, such as those set forth above, can be used in place of beads or microspheres exemplified for these embodiments.

In some embodiments, polymer probes such as nucleic acids or peptides can be synthesized by sequential addition of monomer units directly on a solid support such as a bead or slide surface. Methods known in the art for synthesis of a variety of different chemical compounds on solid supports can be used in the invention, such as methods for solid phase synthesis of peptides, organic moieties, and nucleic acids. Alternatively, probes can be synthesized first, and then covalently attached to a solid support. Probes can be attached to functional groups on a solid support. Functionalized solid supports can be produced by methods known in the art and, if desired, obtained from any of several commercial suppliers for beads and other supports having surface chemistries that facilitate the attachment of a desired functionality by a user. Exemplary surface chemistries that are useful in the invention include, but are not limited to, amino groups such as aliphatic and aromatic amines, carboxylic acids, aldehydes, amides, chloromethyl groups, hydrazide, hydroxyl groups, sulfonates or sulfates. If desired, a probe can be attached to a solid support via a chemical linker. Such a linker can have characteristics that provide, for example, stable attachment, reversible attachment, sufficient flexibility to allow desired interaction with a genome fragment having a typable locus to be detected, or to avoid undesirable binding reactions. Further exemplary methods that can be used in the invention to attach polymer probes to a solid support are described in Pease et al., Proc. Natl. Acad. Sci. USA 91(11):5022-5026 (1994); Khrapko et al., Mol Biol (Mosk) (USSR) 25:718-730 (1991); Stimpson et al., Proc. Natl. Acad. Sci. USA 92:6379-6383 (1995) or Guo et al., Nucleic Acids Res. 22:5456-5465 (1994). Such attachment methods can also be used to attach other nucleic acids, such as small RNA molecules, to solid supports during separation methods. In this regard, reactive groups such as crosslinking moieties can be selectively added to the 5′ phosphate of a small RNA molecule using methods exemplified herein with regard to adding a label to small RNA.

In embodiments including bead-based arrays, the arrays can be made, for example, by adding a solution or slurry of the beads to a substrate containing attachment sites for the beads. A carrier solution for the beads can be a pH buffer, aqueous solvent, organic solvent, or mixture. Following exposure of a bead slurry to a substrate, the solvent can be evaporated, and excess beads removed. Beads can be loaded into the wells of a substrate, for example, by applying energy such as pressure, agitation or vibration, to the beads in the presence of the wells. Methods for loading beads onto array substrates that can be used in the invention are described, for example, in U.S. Pat. No. 6,355,431.

Probes or particles having attached probes can be randomly deposited on a substrate and their positions in the resulting array determined by a decoding step. This can be done before, during or after the use of the array to detect small RNA molecules or other nucleic acids. In embodiments where the placement of probes is random, a coding or decoding system can be used to localize and/or identify the probes at each location in the array. This can be done in any of a variety of ways, as is described, for example, in U.S. Pat. No. 6,355,431, WO 03/002979 or Gunderson et al., Genome Res. 14:870-877 (2004). As will be appreciated by those in the art, a random array need not necessarily be decoded. In this embodiment, beads or probes can be attached to an array substrate, and a detection assay performed to determine the presence of any or all targets independent of identifying which targets are present.

A useful method for making probe arrays is photolithography-based polymer synthesis. For example, Affymetrix® GeneChip® arrays can be synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. patent application Ser. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Application Nos. PCT/US99/00730 (International Publication No. WO 99/36760) and PCT/US01/04285.

Using VLSIPS™, a GeneChip array can be manufactured by reacting the hydroxylated surface of a 5-inch square quartz wafer with silane. Linkers can then be attached to the silane molecules. The distance between these silane molecules determines the probes' packing density, allowing arrays to hold over 500,000 probe locations, or features, within a mere 1.28 square centimeters. Millions of identical DNA molecules can be synthesized at each feature using a photolithographic process in which masks, carrying 18 to 20 square micron windows that correspond to the dimensions of individual features, are placed over the coated wafer. When ultraviolet light is shone over the mask in the first step of synthesis, the exposed linkers become deprotected and are available for nucleotide coupling. Once the desired features have been activated, a solution containing a single type of deoxynucleotide with a removable protection group can be flushed over the wafer's surface. The nucleotide attaches to the activated linkers, initiating the synthesis process. A capping step can be used to truncate unreacted linkers (or polynucleotides in subsequent step). In the next synthesis step, another mask can be placed over the wafer to allow the next round of deprotection and coupling. The process is repeated until the probes reach their full length, usually 25 nucleotides. However, probes having other lengths such as those set forth elsewhere herein can also be attached at each feature. Once the synthesis is complete, the wafers can be deprotected, diced, and the resulting individual arrays can be packaged in flowcell cartridges.

A spotted array can also be used in a method of the invention. An exemplary spotted array is a CodeLink™ Array available from Amersham Biosciences. CodeLink™ Activated Slides are coated with a long-chain, hydrophilic polymer containing amine-reactive groups. This polymer is covalently crosslinked to itself and to the surface of the slide. Probe attachment can be accomplished through covalent interaction between the amine-modified 5′ end of the oligonucleotide probe and the amine reactive groups present in the polymer. Probes can be attached at discrete locations using spotting pens. Useful pens are stainless steel capillary pens that are individually spring-loaded. Pen load volumes can be less than about 200 nL with a delivery volume of about 0.1 nL or less. Such pens can be used to create features having a spot diameter of, for example, about 140-160 μm. In a preferred embodiment, nucleic acid probes at each spotted feature can be 30 nucleotides long. However, probes having other lengths such as those set forth elsewhere herein can also be attached at each spot.

An array that is useful in the invention can also be manufactured using inkjet printing methods such as SurePrint™ Technology available from Agilent Technologies. Such methods can be used to synthesize oligonucleotide probes in situ or to attach pre-synthesized probes having moieties that are reactive with a substrate surface. A printed microarray can contain 22,575 features on a surface having standard slide dimensions (about 1 inch by 3 inches). Typically, the printed probes are 25 or 60 nucleotides in length. However, probes having other lengths such as those set forth elsewhere herein can also be printed at each location.

If desired, nucleic acid probes can be attached to substrates such that they have a free 3′ end for modification by enzymes or other agents. Those skilled in the art will recognize that methods exemplified above in regard to synthesis of nucleic acids in the 3′ to 5′ direction can be modified to produce nucleic acids having free 3′ ends. For example, synthetic methods known in the art for synthesizing nucleic acids in the 5′ to 3′ direction and having 5′ attachments to solid supports can be used in an inkjet printing or photolithographic method. Furthermore, in situ inversion of substrate attached nucleic acids can be carried out such that 3′ substrate-attached nucleic acids become attach to the substrate at their 5′ end and detached at their 3′ end, for example, using methods described in Kwiatkowski et al., Nucl. Acids Res. 27:4710-4714 (1999).

An array of arrays or a composite array having a plurality of individual arrays that is configured to allow processing of multiple samples can be used in the invention. Such arrays allow multiplex detection of small RNA molecules or other nucleic acids. Exemplary composite arrays that can be used in the invention, for example, in multiplex detection formats include one component systems and two component systems as described in U.S. Pat. No. 6,429,027 and U.S. Pat. App. Pub. No. 2002/0102578. A one component system includes a first substrate having a plurality of assay locations each containing an individual array. For example, one or more wells of a microtiter plate can serve as assay locations and can each contain an array of probes. A two component system includes a first component having an attached array which can be contacted with an assay location, such as a well, of a second component. For example, a first component can include one or more posts each having an array on its end and the first component can be configured such that each array fits within an individual well of a second component such as a microtiter plate. Thus, for some applications the number of individual arrays is set by the size of the microtiter plate used including, for example, 96 well, 384 well and 1536 well microtiter plates corresponding to at most 96, 384 or 1536 individual arrays, respectively. Other barriers that can be used to physically separate assay locations include, for example, hydrophobic regions that will deter flow of aqueous solvents, hydrophilic regions that will deter flow of a polar or hydrophobic solvents, a gasket or membrane or combination of these barriers. Further exemplary enclosures that are useful in the invention are described in WO 02/00336, U.S. Pat. App. Pub. No. 02/0102578 or the references cited previously herein in regard to different types of arrays.

The size of an array used in the invention can vary depending on the probe composition and desired use of the array. Arrays useful in the invention can have complexity that ranges from about 2 different probes to many millions, billions or higher. The density of an array can be from 2 to as many as a billion or more different probes per square cm. Very high density arrays are useful in the invention including, for example, those having at least about 10,000,000 probes/cm², including, for example, at least about 100,000,000 probes/cm², 1,000,000,000 probes/cm², up to about 2,000,000,000 probes/cm² or higher. High density arrays can also be used including, for example, those in the range from about 100,000 probes/cm² to about 10,000,000 probes/cm². Moderate density arrays useful in the invention can range from about 10,000 probes/cm² to about 100,000 probes/cm². Low density arrays are generally less than about 10,000 probes/cm².

Those skilled in the art will recognize that specificity of hybridization is generally increased as the length of nucleic acid primers or probes is increased. Thus, a longer nucleic acid can be used, for example, to increase specificity or reproducibility of replication or hybridization, if desired. Accordingly, a nucleic acid used in a method of the invention can be at least 5, 6, 7, 8, 9, 10, 11, 12, 13, 14, 15, 16, 17, 18, 19, 20, 25, 30, 35, 40, 50, 60, 70, 80, 90, 100, 200, 300, 400, 500 or more nucleotides long.

Useful substrates for an array or other solid phase support include, but are not limited to, glass; modified glass; functionalized glass; plastics such as acrylics, polystyrene and copolymers of styrene and other materials, polypropylene, polyethylene, polybutylene, polyurethanes, Teflon, or the like; polysaccharides; nylon; nitrocellulose; resins; silica; silica-based materials such as silicon or modified silicon; carbon; metal; inorganic glass; optical fiber bundles, or any of a variety of other polymers. Useful substrates include those that allow optical detection, for example, by being translucent to energy of a desired detection wavelength and/or do not themselves produce appreciable background fluorescence at a particular detection wavelength.

In a particular embodiment, an array substrate can be an optical fiber bundle or array, as is generally described in U.S. Ser. No. 08/944,850, U.S. Pat. No. 6,200,737; WO9840726, and WO9850782. Each optical fiber can have an individual associated particle, the particle being covalently attached to a probe as is generally described in U.S. Pat. Nos. 6,023,540 and 6,327,410. For example, each fiber end can be etched to form a discrete site to which a bead is associated. Similarly other substrates described herein can contain discrete sites for attachment of probes or association of probe bearing particles. For example, the surface of a substrate can be modified to contain wells, or depressions. This can be done using a variety of techniques, including, but not limited to, photolithography, stamping techniques, molding techniques or microetching techniques. Those skilled in the art will know or be able to determine an appropriate technique based on the composition and shape of the substrate. The sites of an array of the invention need not be discrete sites. For example, it is possible to use a uniform surface of adhesive or chemical functionalities that allows the attachment of probes or particles at any position. Furthermore, a physical barrier, film or membrane can be used over the probes or particles to maintain association with sites and/or protect the probes from degradation.

The sequence of a small RNA molecule from a biological isolate can be determined based on the location or other identifying characteristic of the probe to which it binds. In embodiments including use of a probe array, a plurality of small RNA molecules in a biological isolate can be detected simultaneously allowing efficient determination of the sequences for a plurality of small RNA molecules. Hybridization of an extended small RNA sequence or probe produced from the small RNA sequence to a probe on an array can be detected due to presence of a label in the hybrid. Hybridization to a particular arrayed probe can also be detected by modification of one or both members of the hybrid, for example, using a primer extension method with labeled nucleotides. Exemplary primer extension methods include, single base extension (SBE) or allele specific primer extension (ASPE) and can be carried out as described in U.S. Ser. No. 10/871,513.

A method of distinguishing a small RNA can include quantitating the level of small RNA of a particular sequence in a biological isolate. The level of a small RNA of a particular sequence can be the number or concentration of small RNA molecules having the sequence. Thus, the value can be an absolute value. In particular embodiments, quantitation can be based on a relative value. For example, the amount of a small RNA having a particular sequence can be determined as an amount of signal relative to the amount of signal for another nucleic acid in the same biological isolate. Quantitation of the level of a small RNA can be used to determine expression levels or the extent of RNA interference in different cells. For example, levels of small RNA from cells displaying symptoms of a condition can be compared with levels of small RNA from cells that do not display the condition to determine correlation between RNAi and the condition.

An exemplary condition that can be correlated with expression of one or more small RNAs is cancer (see for example, Calin et al., Proc. Natl. Acad. Sci. USA 101:2999-3004 (2004)). Thus, the invention provides a method of diagnosing susceptibility to a cancer or prognosis of outcome for treatment of cancer.

The prognostic methods of the invention are useful for determining if a patient is at risk for recurrence. Cancer recurrence is a concern relating to a variety of types of cancer. For example, of patients undergoing complete surgical removal of colon cancer, 25-40% of patients with stage II colon carcinoma and about 50% of patients with stage III colon carcinoma experience cancer recurrence. One explanation for cancer recurrence is that patients with relatively early stage disease, for example, stage II or stage III, already have small amounts of cancer spread outside the affected organ that were not removed by surgery. These cancer cells, referred to as micrometastases, cannot typically be detected with currently available tests.

The prognostic methods of the invention can be used to identify surgically treated patients likely to experience cancer recurrence so that they can be offered additional therapeutic options, including preoperative or postoperative adjuncts such as chemotherapy, radiation, biological modifiers and other suitable therapies. The methods are especially effective for determining the risk of metastasis in patients who demonstrate no measurable metastasis at the time of examination or surgery.

The prognostic methods of the invention also are useful for determining a proper course of treatment for a patient having cancer. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or adjust the span of time during which the patient is treated. As described herein, the diagnosis or prognosis of cancer state is typically correlated with expression levels for one or more small RNA molecule.

Exemplary cancers that can be evaluated using a method of the invention include, but are not limited to hematoporetic neoplasms, Adult T-cell leukemia/lymphoma, Lymphoid Neoplasms, Anaplastic large cell lymphoma, Myeloid Neoplasms, Histiocytoses, Hodgkin Diseases (HD), Precursor B lymphoblastic leukemia/lymphoma (ALL), Acute myclogenous leukemia (AML), Precursor T lymphoblastic leukemia/lymphoma (ALL), Myclodysplastic syndromes, Chronic Mycloproliferative disorders, Chronic lymphocytic leukemia/small lymphocytic lymphoma (SLL), Chronic Myclogenous Leukemia (CML), Lymphoplasmacytic lymphoma, Polycythemia Vera, Mantle cell lymphoma, Essential Thrombocytosis, Follicular lymphoma, Myelofibrosis with Myeloid Metaplasia, Marginal zone lymphoma, Hairy cell leukemia, Hemangioma, Plasmacytoma/plasma cell myeloma, Lymphangioma, Glomangioma, Diffuse large B-cell lymphoma, Kaposi Sarcoma, Hemanioendothelioma, Burkitt lymphoma, Angiosarcoma, T-cell chronic lymphocytic leukemia, Hemangiopericytoma, Large granular lymphocytic leukemia, head & neck cancers, Basal Cell Carcinoma, Mycosis fungoids and sezary syndrome, Squamous Cell Carcinoma, Ceruminoma, Peripheral T-cell lymphoma, Osteoma, Nonchromaffin Paraganglioma, Angioimmunoblastic T-cell lymphoma, Acoustic Neurinoma, Adenoid Cystic Carcinoma, Angiocentric lymphoma, Mucoepidermoid Carcinoma, NK/T-cell lymphoma, Malignant Mixed Tumors, Intestinal T-cell lymphoma, Adenocarcinoma, Malignant Mesothelioma, Fibrosarcoma, Sarcomotoid Type lung cacer, Osteosarcoma, Epithelial Type lung cancer, Chondrosarcoma, Melanoma, cancer of the gastrointestinal tract, olfactory Neuroblastoma, Squamous Cell Carcinoma, Isolated Plasmocytoma, Adenocarcinoma, Inverted Papillomas, Carcinoid, Undifferentiated Carcinoma, Malignant Melanoma, Mucoepidermoid Carcinoma, Adenocarcinoma, Acinic Cell Carcinoma, Gastric Carcinoma, Malignant Mixed Tumor, Gastric Lymphoma, Gastric Stromal Cell Tumors, Amenoblastoma, Lymphoma, Odontoma, Intestinal Stromal Cell tumors, thymus cancers, Malignant Thymoma, Carcinids, Type I (Invasive thymoma), Malignant Mesethelioma, Type II (Thymic carcinoma), Non-mucin producing adenocarcinoma, Squamous cell carcinoma, Lymph epithelioma, cancers of the liver and biliary tract, Squamous Cell Carcinoma, Hepatocellular Carcinoma, Adenocarcinoma, Cholangiocarcinoma, Hepatoblastoma, papillary cancer, Angiosarcoma, solid Bronchioalveolar cancer, Fibrolameller Carcinoma, Small Cell Carcinoma, Carcinoma of the Gallbladder, Intermediate Cell carcinaoma, Large Cell Carcinoma, Squamous Cell Carcinoma, Undifferentiated cancer, cancer of the pancreas, cancer of the female genital tract, Squamous Cell Carcinoma, Cystadenocarcinoma, Basal Cell Carcinoma, Insulinoma, Melanoma, Gastrinoma, Fibrosarcoma, Glucagonamoa, Intaepithelial Carcinoma, Adenocarcinoma Embryonal, cancer of the kidney, Rhabdomysarcoma, Renal Cell Carcinoma, Large Cell Carcinoma, Nephroblastoma (Wilm's tumor), Neuroendocrine or Oat Cell carcinoma, cancer of the lower urinary tract, Adenosquamous Carcinoma, Urothelial Tumors, Undifferentiated Carcinoma, Squamous Cell Carcinoma, Carcinoma of the female genital tract, Mixed Carcinoma, Adenoacanthoma, Sarcoma, Small Cell Carcinoma, Carcinosarcoma, Leiomyosarcoma, Endometrial Stromal Sarcoma, cancer of the male genital tract, Serous Cystadenocarcinoma, Mucinous Cystadenocarcinoma, Sarcinoma, Endometrioid Tumors, Speretocytic Sarcinoma, Embyonal Carcinoma, Celioblastoma, Choriocarcinoma, Teratoma, Clear Cell Carcinoma, Leydig Cell Tumor, Unclassified Carcinoma, Sertoli Cell Tumor, Granulosa-Theca Cell Tumor, Sertoli-Leydig Cell Tumor, Disgerminoma, Undifferentiated Prostatic Carcinoma, Teratoma, Ductal Transitional carcinoma, breast cancer, Phyllodes Tumor, cancer of the bones joints and soft tissue, Paget's Disease, Multiple Myeloma, Insitu Carcinoma, Malignant Lymphoma, Invasive Carcinoma, Chondrosacrcoma, Mesenchymal Chondrosarcoma, cancer of the endocrine system, Osteosarcoma, Adenoma, Ewing Tumor, endocrine Carcinoma, Malignant Giant Cell Tumor, Meningnoma, Adamantinoma, Cramiopharlingioma, Malignant Fibrous Histiocytoma, Papillary Carcinoma, Histiocytoma, Follicular Carcinoma, Desmoplastic Fibroma, Medullary Carcinoma, Fibrosarcoma, Anoplastic Carcinoma, Chordoma, Adenoma, Hemangioendothelioma, Memangispericytoma, Pheochromocytoma, Liposarcoma, Neuroblastoma, Paraganglioma, Histiocytoma, Pineal cancer, Rhabdomysarcoms, Pineoblastoma, Leiomyosarcoma, Pineocytoma, Angiosarcoma, skin cancer, cancer of the nervous system, Melanoma, Schwannoma, Squamous cell carcinoma, Neurofibroma, Basal cell carcinoma, Malignant Periferal Nerve Sheath Tumor, Merkel cell carcinoma, Sheath Tumor, Extramamary Paget's Disease, Astrocytoma, Paget's Disease of the nipple, Fibrillary Astrocytoma, Glioblastoma Multiforme, Brain Stem Glioma, Cutaneous T-cell lymphoma, Pilocytic Astrocytoma, Xanthorstrocytoma, Histiocytosis, Oligodendroglioma, Ependymoma, Gangliocytoma, Cerebral Neuroblastoma, Central Neurocytoma, Dysembryoplastic Neuroepithelial Tumor, Medulloblastoma, Malignant Meningioma, Primary Brain Lymphoma, Primary Brain Germ Cell Tumor, cancers of the eye, Squamous Cell Carcinoma, Mucoepidermoid Carcinoma, Melanoma, Retinoblastoma, Glioma, Meningioma, cancer of the heart, Myxoma, Fibroma, Lipoma, Papillary Fibroelastoma, Rhasdoyoma, or Angiosarcoma among others.

The invention provides methods for diagnosing the occurrence of cancer in a patient at risk for cancer. The method involves (a) measuring a level of one or more small RNAs in a neoplastic cell-containing sample from patient at risk for cancer, and (b) comparing the level of the one or more small RNAs in the sample to a reference level, wherein a different level of the one or more small RNAs in the sample correlates with presence of cancer in the patient.

This invention provides methods for determining a prognosis for survival for a cancer patient. One method involves (a) measuring a level of one or more small RNAs in a neoplastic cell-containing sample from the cancer patient, and (b) comparing the level of the one or more small RNAs in the sample to a reference level, wherein a different level of the one or more small RNAs in the sample correlates with increased survival of the patient. The different level can be an increase or decrease of the small RNAs in the sample compared to the reference level.

Another method involves (a) measuring a level of one or more small RNAs in a neoplastic cell-containing sample from a cancer patient, and (b) classifying the patient as belonging to either a first or second group of patients, wherein the first group of patients having a first level of one or more small RNAs is classified as having an increased likelihood of survival compared to the second group of patients having a second level of one or more small RNAs. The level of the one or more small RNAs for the first group can be higher or lower than the level of the one or more small RNAs for the second group.

The invention also provides a method for monitoring the effectiveness of a course of treatment for a patient with cancer. The method involves (a) determining a level of one or more small RNAs in a neoplastic cell containing sample from the cancer patient prior to treatment, and (b) determining the level of one or more small RNAs in a neoplastic cell-containing sample from the patient after treatment, whereby comparison of the level of one or more small RNAs prior to treatment with the level of one or more small RNAs after treatment indicates the effectiveness of the treatment.

As used herein, the term “reference level” refers to a control level of expression of a marker used to evaluate a test level of expression of a biomarker in a neoplastic cell-containing sample of a patient. For example, when the level of one or more small RNAs in the neoplastic cells of a patient are higher than the reference level of one or more small RNAs, the cells will be considered to have a high level of expression of the one or more small RNAs. Conversely, when the level of one or more small RNAs in the neoplastic cells of a patient are lower than the reference level, the cells will be considered to have a low level of expression, or underproduction, of the one or more small RNAs.

A reference level can be determined based on reference samples collected from age-matched normal classes of adjacent tissues, and with normal peripheral blood lymphocytes. The reference level can be determined by any of a variety of methods, provided that the resulting reference level accurately provides a level of a marker, such as one or more small RNAs, above which exists a first group of patients having a different probability of survival than that of a second group of patients having levels of the biomarker below the reference level. The reference level can be determined by, for example, measuring the level of expression of a biomarker in non-tumorous cells from the same tissue as the tissue of the neoplastic cells to be tested. The reference level can also be a level of a biomarker of in vitro cultured cells which can be manipulated to simulate tumor cells, or can be manipulated in any other manner which yields expression levels which accurately determine the reference level. The reference level can also be determined by comparison of the level of a biomarker, such as one or more small RNAs, in populations of patients having the same cancer. This can be accomplished, for example, by histogram analysis, in which an entire cohort of patients are graphically presented, wherein a first axis represents the level of the biomarker, and a second axis represents the number of patients in the cohort whose neoplastic cells express the biomarker at a given level.

Two or more separate groups of patients can be determined by identification of subset populations of the cohort which have the same or similar levels of the biomarker, such as one or more small RNAs. Determination of the reference level can then be made based on a level which best distinguishes these separate groups. A reference level also can represent the levels of two or more small RNAs. The level for two or more small RNAs can be represented, for example, by a ratio of values for levels of each small RNA. The reference level can be a single number, equally applicable to every patient, or the reference level can vary, according to specific subpopulations of patients. For example, older individuals might have a different reference level than younger individuals for the same cancer. In another example, the reference level might be a certain ratio of a biomarker in the neoplastic cells of a patient relative to the biomarker levels in non-tumor cells within the same patient. Thus, the reference level for each patient can be proscribed by a reference ratio of one or more biomarkers, such as one or more small RNAs, wherein the reference ratio can be determined by any of the methods for determining the reference levels described herein.

In a method of staging a cancer it can be useful to apply, in parallel, a series of reference levels, each based on a sample that is derived from a cancer that has been classified based on parameters established in the art, for example, phenotypic or cytological characteristics, as representing a particular cancer stage so as to allow comparison to the biological test sample for purposes of staging. In addition, progression of the course of a condition can be determined by determining the rate of change in the level or pattern of one or more small RNAs by comparison to reference levels derived from reference samples that represent time points within an established progression rate. It is understood, that the user will be able to select the reference sample and establish the reference level based on the particular purpose of the comparison.

A method of the invention can be used to determine the prognosis of disease free survival or overall survival. As used herein, the term “disease-free survival” refers to the lack of recurrence of symptoms such as, in the case of cancer, lack of tumor recurrence and/or spread and the fate of a patient after diagnosis, for example, a patient who is alive without tumor recurrence. The phrase “overall survival” refers to the fate of the patient after diagnosis, regardless of whether the patient has a recurrence of symptoms such as, in the case of cancer, tumor recurrence. Tumor recurrence refers to further growth of neoplastic or cancerous cells after diagnosis of cancer. Particularly, recurrence can occur when further cancerous cell growth occurs in the cancerous tissue. Tumor spread refers to dissemination of cancer cells into local or distant tissues and organs, for example, during tumor metastasis. Tumor recurrence, in particular, metastasis, is a significant cause of mortality among patients who have undergone surgical treatment for cancer. Therefore, tumor recurrence or spread is correlated with disease-free and overall patient survival.

Similar methods to those exemplified above for cancer can be used to diagnose or prognose other conditions. For example, the level of one or more small RNA can be correlated with the presence of Fragile X mental retardation. Such methods can be based on known correlations between levels of one or more small RNAs and Fragile X mental retardation as described, for example, in Jin et al., Nat Neurosci. 7:113-7 (2004). Also, the methods can be useful for diagnosing early-onset inherited Parkinson's disease or other diseases that arise due to aberrant gene expression. Thus, the steps exemplified above for cancer can also be used to diagnose these other diseases, to prognose survival rate or to monitor effectiveness of a course of treatment.

In another embodiment, levels of small RNA can be compared for cells that are treated and untreated with a particular agent to determine effect of the agent on RNAi. For quantitation involving a temporal aspect, relative change in the level of a small RNA can be based on the amount of signal for the small RNA at one time compared to its amount at a different time. For example, the level of a small RNA can be quantitated in the same cells at different times before, during or after exposure to particular conditions or agents.

An exemplary agent that can be added to a cell is a small RNA precursor such as a double stranded RNA. Small RNA precursors and methods of administering them to cells are known in the art as described, for example, in Foley et al., PLOS Biology 2(e203)1-16 or Czauderna et al., Nucl. Acids Res. 31:2705-2716 (2003). Typically, double stranded RNAs that are administered to a cell are smaller than 50 nucleotides in length, exemplary lengths including 30 nucleotides or shorter 25 nucleotides or shorter or 20 nucleotides or shorter. Double stranded RNA can be introduced to a cell using known transfection methods such as electroporation and mediation with lipophilic agents such as Oligofectamine™ or TransIt-TKO™. Other useful methods for delivering double stranded RNA to cells are described, for example, in Kim J. Korean Med. Sci. 18:309-318 (2003) and Duxbury et al. J. Surg. Res. 117:339-344 (2004).

The invention further provides a method of identifying a plurality of different small RNAs. The method includes the steps of (a) providing a plurality of different small RNA sequences; (b) adding unique extension sequences to the different small RNA sequences, thereby forming a plurality of extended small RNA sequences; and (c) detecting the extended small RNA sequences, thereby identifying the plurality of different small RNAs.

Methods of making and using small RNA sequences with and without extension sequences are exemplified below for small RNA molecules. However, it will be understood that products of small RNA molecule replication or amplification can be used with similar results. Unless specified otherwise, small RNA sequences are understood to include, for example, small RNA molecules and nucleic acid replicates thereof. Exemplary replicates of a small RNA include a molecule of DNA, RNA or analog of either that is complementary or identical to the small RNA.

A unique extension sequence can be added to a small RNA sequence to increase its length for subsequent detection or to add sequence elements that further distinguish the small RNA sequence from other sequences in a biological isolate. A unique extension sequence is typically different from one or more other extension sequences in a population of extended small RNA sequences such that the extended small RNA sequence can be distinguished from one or more other extended small RNA sequences based, at least in part, on differences between the unique extension sequences. In particular embodiments, the extension sequence can be unique compared to all other extended small RNA sequences in a population. Typically, a unique extension sequence can be distinguished from other sequences in a biological isolate being evaluated based, for example, on determination of sequence differences between two molecules being compared, determination of different lengths between two or more molecules being compared or separation of two or more molecules being compared.

A variety of methods can be used to add an extension sequence to a small RNA sequence, as set forth in further detail below. Depending upon the method used the extension sequence can be a DNA, RNA, or other oligonucleotide such as an analog of a naturally occurring nucleic acid. A nucleic acid analog can have an alternate backbone including, without limitation, phosphoramide (see, for example, Beaucage et al., Tetrahedron 49:1925 (1993); Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 (1986)), phosphorothioate (see, for example, Mag et al., Nucleic Acids Res. 19:1437 (1991); and U.S. Pat. No. 5,644,048), phosphorodithioate (see, for example, Briu et al., J. Am. Chem. Soc. 111:2321 (1989)), O-methylphophoroamidite linkages (see, for example, Eckstein, Oligonucleotides and Analogues: A Practical Approach, Oxford University Press), and peptide nucleic acid backbones and linkages (see, for example, Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996)). Other analog structures include those with positive backbones (see, for example, Dempcy et al., Proc. Natl. Acad. Sci. USA 92:6097 (1995); non-ionic backbones (see, for example, U.S. Pat. Nos. 5,386,023; 5,637,684; 5,602,240; 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Left. 4:395 (1994); Jeffs et al., J. Biomolecular NMR 34:17 (1994); Tetrahedron Lett. 37:743 (1996)) and non-ribose backbones, including, for example, those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Analog structures containing one or more carbocyclic sugars are also useful in the methods and are described, for example, in Jenkins et al., Chem. Soc. Rev. (1995) pp169-176. Several other analog structures that are useful in the invention are described in Rawls, C & E News Jun. 2, 1997 page 35. Similar analogs can be used in a probe or other nucleic acid of the invention.

A nucleic acid or nucleic acid analog used in the invention can include native or non-native bases or both. Native deoxyribonucleic acid bases include adenine, thymine, cytosine or guanine and native ribonucleic acid bases include uracil, adenine, cytosine or guanine. Exemplary non-native bases that can be used in the invention include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like.

The length of an extension sequence can be chosen to suit a particular embodiment of the methods. For example, detection methods based on ligation or extension-ligation such as those set forth below can benefit from the use of extension sequences that are at least about 10 nucleotides in length so that the extended sequence provides a target of sufficient length for paired probes to hybridize with a desired level of specificity. Those skilled in the art will recognize that longer targets can be employed in such assays to favor increased specificity of detection. Accordingly, an extension sequence can be at least about 15, 20, 25, 30, 40 or 50 or more nucleotides long. The maximum length of an extension sequence can be selected based on any number of considerations including, for example, the cost of producing molecules having the extended sequences or desired properties of molecules having the extended sequences. Accordingly, an extension sequence can be at most 70, 100, 120, 150 or more nucleotides long.

An extension sequence can be unique compared to other extension sequences in a population of extended sequences due to the presence of a unique sequence segment. In addition to a unique sequence segment, each extended sequence in a population can further include one or more segments that are common to other extended sequences in the population. Several common sequence segments that can be included in a extension sequence are described below in the context of various methods for making and detecting extended small RNA sequences and include, for example, universal priming sites, adapter sequences and universal capture sequences.

In particular embodiments, extension sequences can be added to a plurality of small RNA sequences by hybridization. For example, an extension sequence can be added by contacting a biological isolate containing a small RNA sequence with a plurality of different extension sequences having first portions that are complementary to the small RNA sequences and second portions that form single stranded overhangs. The single stranded overhangs can be detected using methods set forth herein. The overhangs can occur at the 5′ end or the 3′ end or at both ends of an extension sequence. An overhang can also occur at one end of the small RNA in a small RNA:extension sequence hybrid. If desired, the first portions of the extension sequences can be covalently bound to the small RNA molecules to stabilize the hybrids, for example, using a chemical cross linking reagent such as psoralen or a photoactivated cross linking reagent. Thus, an extended small RNA sequence can be a double stranded nucleic acid sequence in which a unique extension sequence is part of the oligonucleotide complementing the small RNA sequence.

An extended small RNA sequence can contain one type of nucleic acid species or a mixture of nucleic acid species. For example, an extended small RNA sequence having a mixture of species can contain DNA and RNA segments. The different segments can occur on the same strand, for example, as a DNA-RNA fusion or on different strands, for example, as a DNA:RNA hybrid. Although exemplified for DNA and RNA, those skilled in the art will recognize that mixtures including other nucleic acid species and analogs thereof can also be used in the invention.

In further embodiments, extension sequences can be added to a plurality of small RNA sequences by ligation. For example, a plurality of small RNAs can be hybridized with a plurality of different bridging oligonucleotides having first portions that are complementary to the small molecule sequences and second portions that complement the unique extension sequences. Oligonucleotides having the extension sequences can be hybridized to the second portions of the bridging oligonucleotides and ligated to the small RNAs. A bridging oligonucleotide or other extension sequence can include unique sequence segments or a common sequence segment or both as desired.

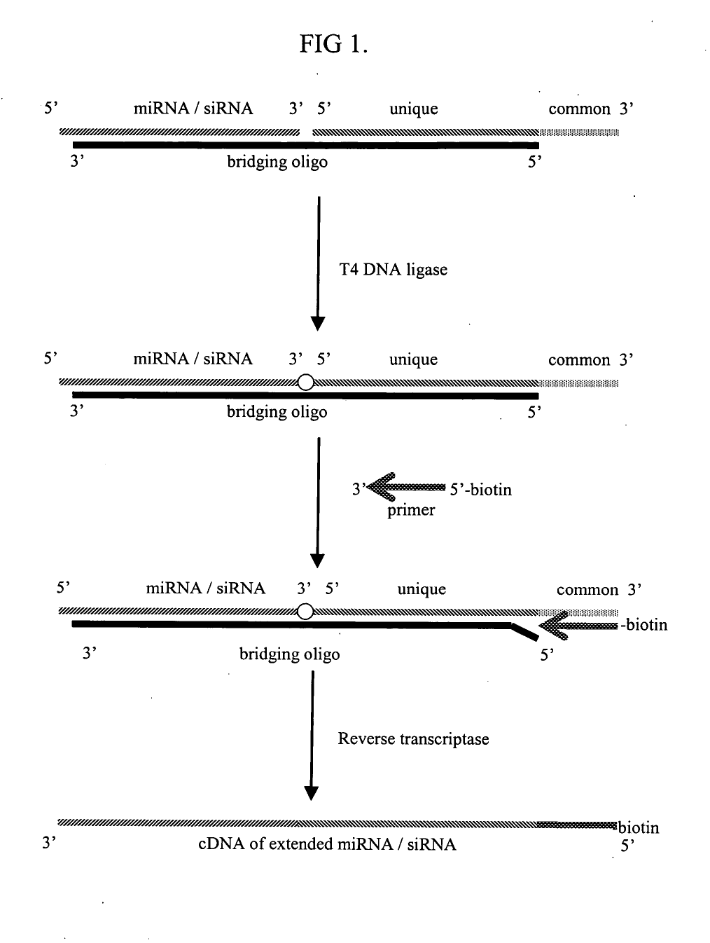

A diagrammatic example of using an extension sequence that acts as a bridging oligonucleotide is shown in FIG. 1. In the diagrammatic example, a DNA bridging oligonucleotide (oligo) hybridizes with a small RNA molecule (miRNA/siRNA) such that the bridging oligo has a 5′ overhang. The 5′ overhang hybridizes with a second DNA molecule having a unique sequence and a common primer site. Although the 5′ end of the bridging oligo is shown to hybridize at the junction of the unique and common sequences it will be understood that the 5′ end can extend into the common sequence or, alternatively, can hybridize within the unique sequence portion such that the bridging oligo hybridizes to only a portion of the unique sequence region. The small RNA molecule and the second DNA molecule are ligated by T4 DNA ligase to form an extended small RNA. The extended small RNA is then annealed to a biotinylated primer at the common primer site and reverse transcribed to form a cDNA complement of the extended small RNA. The cDNA complement can be isolated from other reaction components via the biotin label, if desired. The cDNA complement can also be used for detection of the small RNA sequence. Detection can include one or more of the steps set forth herein including, for example, amplification, array hybridization or replication to form detectable signal probes as occurs in assays such as oligonucleotide ligation amplification, extension-ligation (GoldenGate™), rolling circle and other assays set forth herein.

A small RNA sequence and separate extension sequence can be contiguous when hybridized to a bridging oligonucleotide such that the small RNA sequence and extension sequence can be ligated by a ligase enzyme or chemical crosslinking agent. Alternatively, a small RNA sequence and extension sequence can be separated by a gap of one or more nucleotide positions when hybridized to a bridging oligonucleotide. The gap can be spanned by a chemical crosslinking agent of sufficient length. If desired, the gap can be filled using an extension-ligation reaction in which nucleotides that base-pair with the bridging oligonucleotide are added to the 3′ end of the small RNA sequence or extension sequence followed by ligation of the extended sequence to the 5′ end of the now contiguous sequence. Extension-ligation reactions can be carried out using a polymerase and ligase or other agents as described, for example, in U.S. Pat. No. 6,355,431 B1 and U.S. App. Pub. No. 03/0,211,489.

A bridging oligonucleotide or other oligonucleotide can be hybridized with a small RNA such that overhangs are formed at both ends. For example, a bridging oligonucleotide can hybridize to a small RNA such that the 5′ end of the bridging oligonucleotide has unpaired bases that form an overhang and the 5′ end of the small RNA has unpaired bases also forming an overhang. The presence of two overhangs can be used, for example, to prevent unwanted blunt end ligation and concatemer formation in embodiments such as that exemplified in FIG. 1. Those skilled in the art will recognize that other configurations can be used to prevent unwanted blunt end ligation. Thus, either the bridging oligonucleotide or small RNA can form an overhang at the end opposite the end that is ligated to an extension sequence.

Extension sequences can be added to a plurality of small RNA sequences by polymerase extension. For example, a plurality of small RNAs can be hybridized with a plurality of different bridging oligonucleotides having first portions that are complementary to the small molecule sequences and second portions that form 5′ overhangs. Oligonucleotides having the extension sequences can be synthesized by polymerase extension of the small RNA sequence using the second portions of the bridging oligonucleotides as templates. Thus, a DNA-based or RNA-based extension can be added using a DNA polymerase with deoxyribonucleotides or using an RNA polymerase with ribonucleotides, respectively.

As set forth above, a small RNA molecule made using a method of the invention can have a DNA extension. For example, a DNA molecule can be ligated to a small RNA molecule to form an extended small RNA sequence. DNA and RNA can be ligated using a ligase capable of recognizing DNA and RNA such as T4 DNA ligase or a chemical crosslinker. In other embodiments, a small RNA molecule can be extended by a DNA polymerase to incorporate deoxyribonucleotides such that an RNA-DNA fusion product is formed. For example, DNA Pol I is useful for extending an RNA primer with deoxyribonucleotides.

As exemplified above, an extension sequence need not be located on the same strand as a small RNA sequence. Rather, the extension sequence can be located on the strand that complements the small RNA sequence. If desired, embodiments can be used where the extension sequence is not located on a strand that complements the small RNA sequence. Accordingly, an extension sequence can occur in the same strand as the small RNA sequence. Furthermore, an extension sequence can be added to the 5′ end or 3′ end of a small RNA sequence. Those skilled in the art will know or be able to determine appropriate enzymes, sequence configurations and other relevant conditions in order to add an extension sequence to a particular end of a small RNA sequence in a method of the invention.

As exemplified briefly above in regard to the embodiment diagrammed in FIG. 1, an extended small RNA sequence can include a common sequence that functions as a universal priming site. A universal priming site can be placed in extended small RNA sequences made by any of a variety of methods described herein. The universal priming site can be located downstream (on the 3′ side of) sequences that are to be replicated or amplified. Thus, a universal priming site can be located 3′ of a unique extension sequence or a small RNA sequence or both. Accordingly, a method of the invention can include a step of amplifying an extended small RNA sequence using a universal primer that hybridizes to a universal priming site present in the extended small RNA sequence. Furthermore, a small RNA sequence that is produced by replication or amplification of an extended small RNA molecule can also include one or more universal priming site and can, therefore, be further amplified or replicated in a method of the invention. Amplification can be carried out using methods known in the art including, for example, the polymerase chain reaction (PCR), strand displacement amplification (SDA), ligase chain reaction (LCR) or nucleic acid sequence based amplification (NASBA).

Replication of an extended small RNA sequence can be carried out using any of a variety of polymerases known in the art under conditions known in the art. For example, small RNA sequences that contain ribose sugars can be reverse transcribed with a polymerase having reverse transcriptase activity (RT). Exemplary RTs that can be used in a method of the invention include, but are not limited to, those from retroviruses such as avian myoblastosis virus (AMV) RT, Moloney murine leukemia virus (MMLV) RT, HIV-1 RT, or Rouse sarcoma virus (RSV) RT. Generally, a reverse transcription reaction used in a method of the invention will include a template having at least a portion of ribose backbone, one or more dNTPs and a nucleic acid primer with a 3′ OH group.

Extended small RNA sequences that occur in DNA molecules, such as replicates or amplicons of extended small RNA molecules, can be further amplified or replicated using a DNA polymerase or RNA polymerase. Exemplary RNA polymerases that are useful in the invention include, but are not limited to, T7 RNA polymerase and T3 RNA polymerase. Exemplary DNA polymerases include, without limitation, DNA polymerase I, Bst I Polymerase, the Klenow fragment of DNA polymerase I, T5 DNA polymerase, Phi29 DNA polymerase or Taq polymerase. Furthermore functional variants of naturally occurring polymerases can be used including, for example, SEQUENASE™ 1.0 and SEQUENASE™ 2.0 (U.S. Biochemical), Thermosequenase™ (Taq with the Tabor-Richardson mutation), those lacking exonuclease function (exo-variants) and others known in the art or described herein. Useful polymerase as well as conditions for their use are described, for example, in Eun, Enzymology Primer for Recombinant DNA Technology, Academic Press, San Diego (1996). The polymerases described above can also be used in extension-ligation and polymerase extension methods described herein.

In some embodiments, target amplification-based detection techniques can be used in which an extended small RNA sequence is replicated to form detectable signal probes, thereby allowing a small number of target molecules to result in a large number of signaling probes, that then can be detected. Probe amplification-based strategies include, for example, oligonucleotide ligation amplification (OLA), extension-ligation, rolling circle amplification (RCA), the ligase chain reaction (LCR), cycling probe technology (CPT), invasive cleavage techniques such as Invader™ technology, Q-Beta replicase (QβR) technology or sandwich assays. Such techniques can be carried out, for example, under conditions described in U.S. Pat. App. Pub. Nos. 03/0207295 and 03/0108900 and U.S. Pat. No. 6,355,431 B1, or as set forth below.

Detection with oligonucleotide ligation amplification (OLA) involves the template-dependent ligation of two smaller probes into a single long probe, using a template having a small RNA sequence such as an extended small RNA sequence. In a particular embodiment, a single-stranded target sequence includes a first target domain and a second target domain, which are adjacent and contiguous. A first OLA probe and a second OLA probe can be hybridized to complementary sequences of the respective target domains. The two OLA probes are then covalently attached to each other to form a modified probe. In embodiments where the probes hybridize contiguously with each other, covalent linkage can occur via a ligase. In one embodiment one of the ligation probes can be attached to a surface such as an array or a particle. In another embodiment both ligation probes can be in solution and the ligated probe subsequently detected, for example, by hybridization to a surface attached probe.

Alternatively, an extension-ligation assay can be used wherein two smaller probes hybridize to template having a small RNA sequence such as an extended small RNA sequence such that the two probes are non-contiguous. Thus, a gap of one or more nucleotide positions occurs between the hybridized probes. One or more nucleotides can be added to the 3′ end of one of the probes to fill the gap and the extended probe can be ligated to the second probe. Extension and ligation can be carried out using, for example, a polymerase and ligase, respectively. If desired, hybrids between modified probes and targets can be denatured, and the process repeated for amplification leading to generation of a pool of ligated probes. As above, these extension-ligation probes can be, but need not be, attached to a surface such as an array or a particle. Extension-ligation assay can be carried out under the GoldenGate™ protocol as described, for example, in Shen et al., Genetic Engineering News 23 (2003). Further conditions for ligation and extension-ligation assays that are useful in the invention are described, for example, in U.S. Pat. App. Pub. Nos. 03/0207295 and 03/0108900 and U.S. Pat. No. 6,355,431 B1.

OLA and extension-ligation assays result in linear amplification of an extended small RNA sequence. If desired, exponential amplification can be carried out using a double stranded, extended small RNA sequence and modified versions of OLA or extension-ligation. OLA is referred to as the ligation chain reaction (LCR) when double-stranded targets are used. In LCR, two sets of probes are used: one set as outlined above for one strand of the target, and a separate set for the other strand of the target. Repeated cycles of hybridization, ligation and denaturation result in exponential amplification of the extended small RNA sequence. Similarly extension-ligation probes can be used for exponential amplification of a template having a small RNA sequence using repeated cycles of hybridization, extension, ligation and denaturation.

A template having a small RNA sequence such as an extended small RNA sequence can be detected in a method of the invention using rolling circle amplification (RCA). In a first embodiment, a single probe can be hybridized to a template target such that the probe is circularized while hybridized to the target. Each terminus of the probe hybridizes adjacently on the target nucleic acid. The circular probe can be ligated, denatured from the target and a polymerase added resulting in amplification of the circular probe. Following RCA the amplified circular probe can be detected, for example, by hybridization to a solid-phase probe or array. A circular probe used in the invention can further include other characteristics such as an adaptor sequence, restriction site for cleaving concatamers, a label sequence, or a priming site for priming the RCA reaction. Rolling-circle amplification can be carried out under conditions such as those generally described in U.S. Pat. App. Pub. Nos. 03/0207295 and 03/0108900; U.S. Pat. No. 6,355,431 B1; Baner et al. Nuc. Acids Res. 26:5073-5078 (1998); Barany, F. Proc. Natl. Acad. Sci. USA 88:189-193 (1991); or Lizardi et al. Nat Genet. 19:225-232 (1998).

It will be understood that, the detection assays set forth herein can be used in various combinations or with various modifications to suit a particular application of the invention. For example, detection can include OLA followed by RCA. In this embodiment, first and second probes are ligated when hybridized to an extended small RNA sequence. The small RNA sequence can then be removed and the ligated probe product, hybridized with a circular probe that is amplified via an RCA reaction. In another embodiment, RCA probes can hybridize to form a gap that is closed by extension-ligation.

Although detection assay methods that involve ligation of two probes have been exemplified above by use of a ligase enzyme, it will be understood that chemical ligation can be used if desired. In this embodiment, at least one of the probes can include an activatable cross-linking agent that upon activation, results in a chemical cross-link with the adjacent probe. The activatable group can include any moiety that will allow cross-linking of the probes, and include groups activated chemically, photonically or thermally, such as photoactivatable groups. In some embodiments a single activatable group on one of the side chains is enough to result in cross-linking via interaction to a functional group on the other side chain; in alternate embodiments, activatable groups can be included on each side chain. Exemplary methods of chemical ligation that can be used are described, for example, in U.S. Pat. Nos. 5,616,464 and 5,767,259.

In particular embodiments, a detection assay can include invasive cleavage technology. Using such an approach, a template having a small RNA sequence such as an extended small RNA sequence can be hybridized to two distinct probes. The two probes are an invader probe, which is substantially complementary to a first portion of the extended small RNA sequence, and a signal probe, which has a 3′ end substantially complementary to a sequence having a detection position and a 5′ non-complementary end which can form a single-stranded tail. Hybridization of the invader and signal probes near or adjacent to one another on an extended small RNA sequence can form any of several structures useful for detection of the probe-fragment hybrid. Typically, a nuclease that recognizes the structure catalyzes release of the tail which is subsequently detected. Invasive cleavage technology can be used in the invention using conditions and detection methods described, for example, in U.S. Pat. Nos. 6,355,431; 5,846,717; 5,614,402; 5,719,028; 5,541,311; or 5,843,669.

A further detection assay that can be used to identify an extended small RNA sequence is cycling probe technology (CPT). A CPT probe can include two probe sequences separated by a scissile linkage. The CPT probe is substantially complementary to a template having a small RNA sequence such as an extended small RNA sequence and thus will hybridize to it forming a probe-fragment hybrid. Typically the temperature and probe sequence are selected such that the primary probe will bind and shorter cleaved portions of the primary probe will dissociate. A probe-fragment hybrid formed in the methods can be subjected to cleavage conditions which cause the scissile linkage to be selectively cleaved, without cleaving the target sequence, thereby separating the two probe sequences. Cleaved probes produced by a CPT reaction can be detected using methods such as hybridization to an array or other methods set forth herein. CPT methods can be carried out under conditions described, for example, in U.S. Pat. Nos. 5,011,769; 5,403,711; 5,660,988; and 4,876,187, and PCT published applications WO 95/05480; WO 95/1416, and WO 95/00667.

A probe used in a detection assay such as OLA, extension-ligation, RCA, CPT, LCR and others known in the art can include sequences for one or more universal priming sites. Universal priming sites can be used to amplify probes that have been modified in the presence of an appropriate small RNA sequence target. For example, one or more small OLA probe can include a universal priming site such that the ligated OLA probe produced in the presence of a complementary extended small RNA sequence will include universal priming sites that flank the portion of the ligated probe that complements the extended small RNA sequence. PCR can be used to amplify the ligated probe template with universal primers to produce amplicons for subsequent detection. Universal priming sites and methods for their use in the context of detection assays are described, for example, in U.S. Pat. App. Pub. Nos. 03/0207295 and 03/0108900; U.S. Pat. No. 6,355,431 B1. As set forth previously herein, an extension sequence used in a method of the invention can also include a universal priming site.

In particular embodiments, a probe used in a detection assay can include a detectable label. The label can be detected for example at a specific location on a probe array to identify a particular extended small RNA sequence of interest. As set forth above, a probe can be attached to a surface or particle and subsequently modified to incorporate a label in an assay such as those set forth above. For example, a solid-phase probe can be hybridized to an appropriate extended small RNA sequence such that it is contiguous with a soluble labeled probe that is also hybridized to the extended small RNA sequence and the two probes can be subsequently ligated, thereby attaching the label to the solid-phase probe. Alternatively, a probe can be modified in solution and subsequently detected via hybridization to a complementary probe, for example, on an array. It will be understood that a label can be incorporated into a probe using one or more labeled nucleotides added during a probe modification step, for example, using a polymerase.

If desired, a probe or extension sequence can include an adapter sequence, (sometimes referred to in the art as a “zip code” or “address sequence”). An adapter sequence is a nucleic acid that is generally not native to the target sequence, but is added or attached to the target sequence. In particular embodiments, the adapters are hybridization adapters. In this embodiment, adapters are chosen so as to allow hybridization to the complementary capture probes on a surface of universal array. Adapters serve as unique identifiers of the probe and thus of the target sequence. In general, sets of adapters and the corresponding capture probes on arrays are developed to minimize cross-hybridization with both each other and other components of the reaction mixtures, including, for example, sequences for RNA and DNA molecules of a cell being used in the method. An advantage of using universal arrays and adapter sequences is that the content of an array need not be altered to detect different populations of small RNA sequences. Rather, sequences of soluble probes, such as OLA probes, can be selected such that specific adapters are assigned to different small RNA sequences in different biological isolates.

Although the invention is exemplified herein with respect to an array of immobilized probes, those skilled in the art will recognize that other detection formats can be employed as well. For example, the methods set forth herein can be carried out in solution phase rather than solid phase. Accordingly, solution phase probes can replace immobilized probes in the methods set forth above. Solution phase probes can be detected according to properties such as those set forth above in regard to detection labels or detection moieties. For example, probes can have identifiable charge, mass, charge to mass ratio or other distinguishing properties. Such distinguishing properties can be detected, for example, in a chromatography system such as capillary electrophoresis, acrylamide gel, agarose gel or the like, or in a spectroscopic system such as mass spectroscopy.

Throughout this application various publications, patents and patent applications have been referenced. The disclosures of these publications in their entireties are hereby incorporated by reference in this application in order to more fully describe the state of the art to which this invention pertains.

The term “comprising” is intended herein to be open-ended, including not only the recited elements, but further encompassing any additional elements.

Although the invention has been described with reference to the examples provided above, it should be understood that various modifications can be made without departing from the invention. Accordingly, the invention is limited only by the claims.

What is claimed is:

1. A method of detecting a plurality of different small RNAs, comprising
(a) providing a biological isolate comprising mRNA having a 5′ cap structure and a plurality of different small RNA molecules having a 5′ phosphate;
(b) contacting said mixture with a phosphate reactive reagent comprising a label moiety under conditions wherein said label moiety is preferentially added to said 5′ phosphate over said 5′ cap structure, thereby producing a plurality of labeled small RNA;
(c) adding a unique extension sequence to each different small RNA, thereby forming a plurality of extended small RNAs; and
(d) detecting said extended small RNAs, thereby identifying said plurality of different small RNAs.

2. The method of claim 1, wherein said label moiety comprises a ligand.

3. The method of claim 2, wherein said detecting comprises specifically binding said ligand to a receptor.

4. The method of claim 3, wherein said receptor is immobilized to a solid support.

5. The method of claim 1, further comprising separating said labeled small RNA from said mRNA.

6. The method of claim 1, wherein said label moiety comprises a fluorophore.

7. The method of claim 1, wherein said phosphate reactive reagent comprises a carbodiimide and a label moiety having an amino group.

8. The method of claim 1, wherein said labeled small RNA comprises a phosphoramide linkage between said RNA and said label moiety.

9. The method of claim 1, further comprising removing said label moiety from said labeled small RNA after step (b).

10. The method of claim 1, wherein said small RNA comprises microRNA or short interfering RNA.

12. The method of claim 12, wherein said detecting comprises hybridizing said extended small RNAs to an array of probe molecules.

13. The method of claim 1, wherein said adding comprises ligating said unique extension sequence to each of said small RNA sequences.

14. The method of claim 1, wherein said adding comprises hybridizing said unique extension sequence to each of said small RNA sequences.

15. The method of claim 1, wherein said adding comprises synthesis of said unique extension sequence by polymerase extension of said small RNA sequence.

16. The method of claim 1, wherein said unique extension sequence further comprises a universal priming site.

17. The method of claim 1, further comprising amplifying said small RNA sequences using a universal primer that hybridizes to said universal priming site.

18. The method of claim 17, wherein said amplifying is carried out by MMLV reverse transcriptase.

19. The method of claim 1, wherein said unique extension sequence comprises DNA.

20. The method of claim 19, wherein said small RNA sequences comprise RNA and said small RNA sequence and said DNA are ligated by T4 DNA ligase.
